SEQUENCE LISTING (6 sequences)

SEQ ID NO: 1 - DNA, 325 bp, Homo sapiens
ccgcttcttcaggcagtgcctggggcgggagggttggggtgtgggtggctccctaagtcgacactcgtgcggctgcggttccagccccctccccccgccactcaggggcgggaagtggcgggtgggagtcacccaagcgtgactgcccgaggcccctcctgccgcggcgaggaagctccataaaagccctgtcgcgacccgctctctgcaccccatccgctggctctcacccctcggagacgctcgcccgacagcatagtacttgccgcccagccacgcccgcgcgccagccaccgtgagtgctacgacccgtctgtctaggggt

SEQ ID NO: 2 - DNA, 247 bp, Homo sapiens
cccgtccgttgagccttctgtgcctgcagatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcagcgctgcgacactacatcaacctcatcaccaggcagaggtgggtgggaccgcgggaccgattccggga

SEQ ID NO: 3 - DNA, 142 bp, Homo sapiens
acttgctttaaaagacttttttttttccagatatggaaaacgatctagcccagagacactgatttcagacctcttgatgagagaaagcacagaaaatgttcccagaactcggtatgacaaggcttgtgatggggacattgtt

SEQ ID NO: 4 - DNA, 300 bp, Homo sapiens
ccttacatgctttgcttcttatgttttacaggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattcaaaagtgtatcctcctcaatgaaaaatctattacaatagtgaggattattttcgttaaacttattattaacaa

SEQ ID NO: 5 - DNA, 551 bp, Homo sapiens
  CDS (87)..(377)
  sig_peptide (87)..(170)
accccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattc

SEQ ID NO: 6 - protein, 97 aa, Homo sapiens
MLGNKRLGLSGLTLALSLLVCLGALAEAYPSKPDNPGEDAPAEDMARYYSALRHYINLITRQRYGKRSSPETLISDLLMRESTENVPRTRLEDPAMW

(19) United States
(12) Patent Application Publication
(10) Pub. No.: US 2003/0224362 A1
(43) Pub. Date: Dec. 4, 2003

(54) Diagnosis of a person's risk of developing alcoholism

(76) Inventors: Jussi Kauhanen, Kuopio (FI); Matti Karvonen, Turku (FI); Ullamari Pesonen, Turku (FI); Markku Koulu, Turku (FI); Matti Uusitupa, Kuopio (FI)

Correspondence Address: ROTHWELL, FIGG, ERNST & MANBECK, P.C., 1425 K STREET, N.W., SUITE 800, WASHINGTON, DC, 20005, US

(21) Appl. No.: 10/069,129

(22) PCT Filed: Feb. 6, 2001
(86) PCT No.: PCT/FI01/00103

(51) Int. Cl. C12Q 1/68; A61K 31/00

(52) U.S. Cl. 435/6; 514/1

(57) ABSTRACT

The invention relates to methods for diagnosing a person's susceptibility for having a risk for the development of alcoholism. The invention relates further to methods for treating persons diagnosed for having risk for the development of alcoholism in order to prevent the development of said condition. The invention also concerns methods to investigate or screen pharmaceuticals or genetic aims useful in the treatment of said condition, by using an animal model including a transgenic animal.

### FIELD OF THE INVENTION

This invention relates to methods for diagnosing a person's susceptibility for having a risk for the development of alcoholism. The invention relates further to methods for preventing or treating persons diagnosed for having risk for the development of alcoholism, in order to prevent the development of said condition. The invention also concerns methods to investigate or screen pharmaceuticals or genetic aims useful in the prevention or treatment of alcoholism, by using an animal model including a transgenic animal.

### BACKGROUND OF THE INVENTION

The publications and other materials used herein to illuminate the background of the invention, and in particular, cases to provide additional details respecting the practice, are incorporated by reference.

Neuropeptide Y (NPY) is a hexatriocontapeptide amide that is well characterized as a neuromodulator in the central nervous system [Gray and Morley, 1986; Lundberg et al., 1982]. The best known effects of NPY are stimulation of feeding [Clark et al., 1985; Levine and Morley, 1985; Stanley and Leibowitz 1985] and increased energy storage through lipoprotein lipase activation in white adipose tissue [Billington et al., 1991; Billington et al., 1994]. Recent findings in rodents suggest that NPY may also be a potential regulator of ethanol consumption [Ehlers et al., 1998a; Ehlers et al., 1998b; Thiele et al., 1998; Cokerill, 1998; Tecott and Heberlein, 1998]. The preference for alcohol seems to be inversely related to NPY levels in brain [Thiele et al., 1998]. NPY-deficient mice show increased consumption of ethanol, whereas transgenic mice that overexpress a NPY gene have a lower preference for ethanol and are more sensitive to its sedative/hypnotic effects [Thiele et al., 1998]. NPY and ethanol have a similar electrophysiological profile [Ehlers et al., 1998b], and both are known to have anxiolytic properties [Thiele et al., 1998; Heilig et al., 1992; Palmiter et al., 1998; Stewart et al., 1993]. In addition, NPY might influence consumption behaviors through reward effects [Ehlers et al., 1998a; Tecott and Heberlein, 1998]. NPY is expressed in the amygdala and nucleus accumbens, structures of the mesolimbic dopamine system that are thought to mediate the rewarding aspects of food, alcohol and certain drugs [Tecott and Heberlein, 1998; Ault et al., 1993; Jewett et al., 1992]. Despite the circumstantial evidence from animal models, no studies on the role of NPY in the regulation of alcohol consumption in humans have yet been published.

A novel finding of a common polymorphism in the signal peptide of NPY was recently reported [Karvonen et al., 1998]. After screening the entire coding region of the NPY gene for sequence variants, a thymidine(1128) to cytosine(1128) polymorphism(T1128C) was identified, resulting in a substitution of Leu(7) to Pro(7) in the signal peptide part of preproNPY. The Pro (7) in NPY showed a strong association with elevated serum cholesterol levels [Karvonen et al., 1998].

In the present study we found that the Leu (7) to Pro (7) polymorphism in NPY is related to the level of alcohol consumption in an unselected male population sample from the Kuopio Ischemic Heart Disease Risk Factor Study (KIHD) [Salonen, 1988; Lakka et al., 1994].

### SUMMARY OF THE INVENTION

According to one aspect, this invention concerns a method for diagnosing a person's susceptibility for having a risk for the development of alcoholism, said method comprising determining whether said subject has a polymorphism in the signal peptide part of the human preproNPY, said polymorphism comprising the subsitution of the position 7 leucine for proline in the signal peptide part of said preproNPY, said polymorphism being indicative of a risk for the development of alcoholism.

According to a second aspect, the invention concerns a method for treating a person, diagnosed for having a risk for the development of alcoholism, for the prevention of developing alcoholism or for alleviating or curing of alcoholism, comprising administering to said person an effective amount of an agent counteracting the influence of the mutated NPY gene.

According to a third aspect, the invention concerns a method for treating a person, diagnosed for having a risk for the development of alcoholism, for the prevention of developing alcoholism or for alleviating or curing of alcoholism, comprising subjecting the person to specific gene therapy aimed to repair the mutated NPY signal peptide sequence.

According to a fourth aspect, the invention concerns a method to investigate or screen pharmaceuticals or genetic aims useful in the prevention or treatment of alcoholism, by using an animal model including a transgenic animal which carries a human DNA sequence comprising a nucleotide sequence encoding a prepro-neuropeptide Y (preproNPY) or part thereof encoding mature human NPY peptide, where the leucine amino acid in position 7 of the signal peptide part of said preproNPY i) is unchanged or ii) has been replaced by proline.

According to a fifth aspect, the invention concerns a method to investigate or screen pharmaceuticals or genetic aims useful in the prevention or treatment of alcoholism, by using an animal model including a transgenic animal, which carries a DNA sequence comprising a nucleotide sequence encoding otherwise normal mouse NPY sequence or part thereof encoding mature mouse NPY peptide, but in which the nucleotide sequence encoding the mouse signal peptide is replaced by human signal peptide sequence encoding either normal or mutated human signal peptide.

### BRIEF DESCRIPTION OF THE DRAWINGS

FIG. 1a illustrates schematically the molecular structure of the human NPY gene, the preproNPY peptide and the mature NPY peptide,

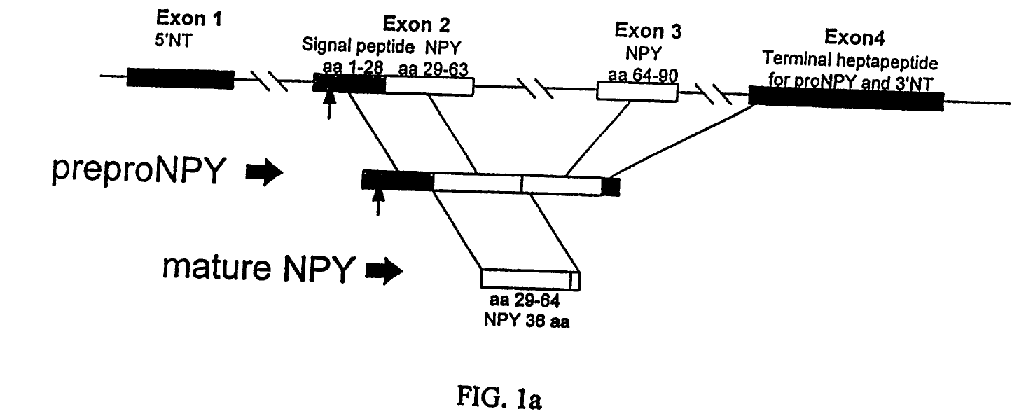

FIG. 1b shows the nucleotide sequence of the human NPY gene. Upper case indicates exonic sequences and lower case intronic sequences. Genbank accession numbers are given in parenthesis. The arrow shows the position in which thymidine (T) of the normal gene is replaced by cytosine (C) to give the mutant gene. The underlined sequence in Exon 2 is the sequence encoding the signal peptide of 28 amino acids (Exon 1 is SEQ ID NO:1, exon 2 is SEQ ID NO:2, exon 3 is SEQ ID NO:3 and exon 4 is SEQ ID NO:4), and

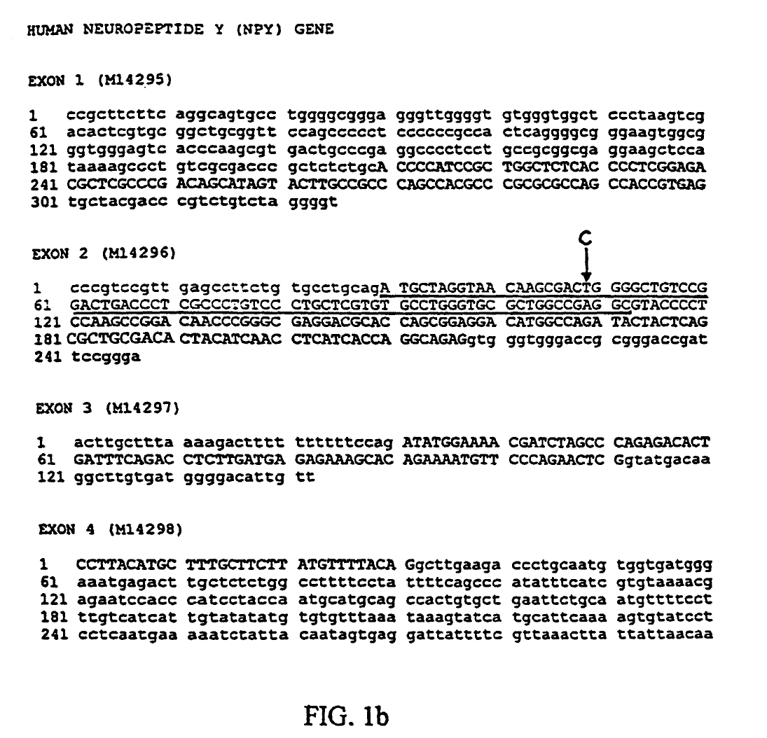

FIG. 1c shows the nucleotide sequence of the human preproNPY mRNA (SEQ ID NO:5, with the protein sequence set forth in SEQ ID NO:6). The arrow shows the position in which thymidine (t) of the normal mRNA is replaced by cytosine (c) to give the mutant mRNA.

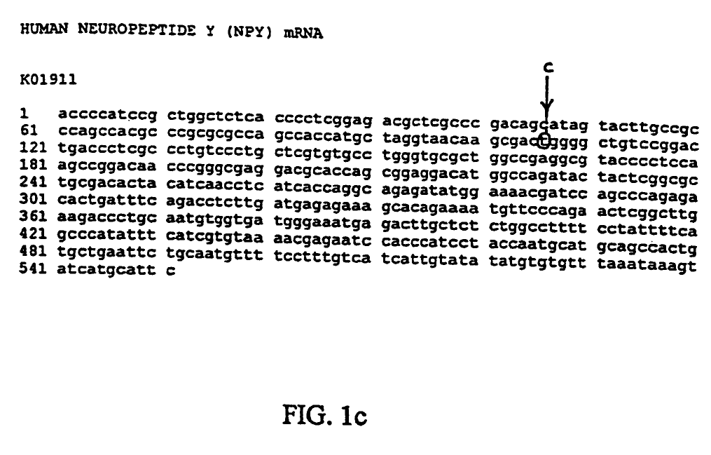

### DETAILED DESCRIPTION OF THE INVENTION

Neuropeptide Y (NPY) is a 36-amino-acid neurotransmitter widely present in the central and peripheral nervous systems. NPY has multiple actions, which control body energy balance and cardiovascular function. We have recently demonstrated that the subjects having Pro7 in the signal peptide of NPY have higher serum cholesterol and apolipoprotein B levels when compared to individuals having wildtype (Leu7/Leu7) signal peptide sequence. Neuropeptide Y (NPY) plays an important role in the hypothalamic regulation of food intake and energy balance. According to recent findings in animals, NPY also appears to be a potent regulator of alcohol consumption. We used recently identified Leu (7) to Pro (7) polymorphism in the signal peptide part of NPY to investigate wheter the NPY system is associated with alcohol consumption in humans. The subjects (N=889) were an ethnically homogenous, unselected population sample of middle-aged men from Eastern Finland. The gene variant producing Pro (7) substitution was associated with a 34% higher average alcohol consumption, even following adjustment for a number of covariates (p=0.03). The proportion of heavy drinkers (over 230 grams of ethanol/week) was also somewhat higher in this group (13.1% vs. 8.2%, p=0.10). Our study provides the first evidence that alcohol preference in humans is likely to be regulated by the NPY system.

The DNA sequence or the mutant signal peptide or said peptide associated with any other cleavage product of preproNPY can be used for screening a subject to determine if said subject is a carrier of a mutant NPY gene.

The determination can be carried out either as a DNA analyse according to well known methods, which include direct DNA sequencing of the normal and mutated NPY gene, allele specific amplification using the polymerase chain reaction (PCR) enabling detection of either normal or mutated NPY sequence, or by indirect detection of the normal or mutated NPY gene by various molecular biology methods including e.g. PCR-single stranded conformation polymorphism (SSCP)-method or denaturing gradient gel electrophoresis (DGGE). Determination of the normal or mutated NPY gene can also be done by using restriction fragment length polymorphism (RFLP)-method, which is particularly suitable for genotyping large number of samples.

The determination can also be carried out at the level of RNA by analysing RNA expressed at tissue level using various methods. Allele spesific probes can be designed for hybridization. Hybridization can be done e.g. using Northern blot, RNase protection assay or in situ hybridization methods. RNA derived from the normal or mutated NPY gene can also be analysed by converting tissue RNA first to cDNA and thereafter amplifying cDNA by an allele spefic PCR-method and carrying out the analysis as for genomic DNA as mentioned above.

Alternatively, the determination can be carried out as an immunoassay where a sample is contacted with an antibody capable of binding the signal peptide or said peptide associated with any other cleavage product of preproNPY.

Antibodies can be raised against normal or mutated preproNPY or more specifically against normal or mutated signal peptide part of the NPY. The production of antibodies can be done in experimental animals in vivo to obtain polyclonal antibodies or in vitro using cell lines to obtain monoclonal antibodies.

A person diagnosed for having a risk for the development of alcoholism can be treated for the prevention of developing said condition by administering to said person an effective amount of an agent counteracting the influence of the mutated NPY gene. This can be done by specific gene therapy aimed to repair the mutated NPY sequence, or by administering pharmacotherapies, which are aimed to modulate synthesis, release or metabolism of the endogenous NPY, or to interact in a specific manner at NPY target sites by modulating effects of NPY with specific NPY receptor proteins. Currently, five different subtypes of NPY receptors have been cloned and characterized (Y1-Y5 receptors) and drug molecules specifically interacting with these NPY receptors have been synthesized. The pharmacotherapy described is not limited to only these named receptors or mechanisms, but also covers other NPY receptors and related mechanisms to be discovered including the secretion of NPY.

The influence of the mutated NPY gene in a patient can be counteracted by using an antisense therapy or gene switching or replacement, which includes targeted correction of disease-related mutation or site-directed inactivation of the mutant allele by homologous recombination.

The antisense therapy refers to methods designed to impair translation through direct interactions with target messenger RNA (mRNA). This can be accomplished by applying a targeted oligonucleotide, which forms Watson-Crick base pairs with the messenger RNA whose function is to be disrupted. The inhibition of gene expression by antisense oligonucleotide depends on the ability of an antisense oligonucleotide to bind a complementary mRNA sequence and prevent the translation of the mRNA. It is possible to correct a single mutant base in a gene by using an oligonucleotide based strategy (Giles et al., 1995; Schwab et al., 1994; Yoon et al., 1996). A short, 7 or 8 bases, oligonucleotide is enough to posses an antisense activity and specificity, which depends greatly on the flanking sequences of the target RNA. Binding should be enough to promote stable binding and RNase H—mediated cleavage.

The influence of the mutated NPY gene is preferably counteracted by using a short, allele specific oligonucleotide, which includes the sequence of mutated part: . . . cga ct/cg ggg . . . . This can be accomplished by using oligonucleotides of various lengths, but all recognizing the mutated base sequence. According to the predicted secondary structure of the preproNPY mRNAs (Schemes 1 and 2), the best target sequence is between −9 and +2 bases around the mutation i.e. sequence targeting to 3′-ac aag cga ctg g-5′. This sequence contains ‘bulbs’ which are known to enhance the binding of oligonucleotide to the target mRNA.

It is possible to use unmodified oligonucleotides, but to increase their stability, nuclease resistance, and penetration to the nucleus, several modifications of oligonucleotide can be used. A relatively large number of modified pyrimidines have been synthesized, mainly C-2, C-4, C-5, and C-6 sites, and incorporated into nucleotides. Also purine analogs can be synthesized and incorporated into oligonucleotides. The 2′ position of the sugar moiety, pentofuranose ring, is substituted with methoxy, propoxy, O-alkoxy or methoxyethoxy groups. A new backbone for oligonucleotides that replace the phosphate or the sugar-phosphate unit has been made, like C-5 propynylpyrimidine-modified phosphothioate oligonucleotides. Also chimeric oligonucleotides with 5′- and 3′-ends are modified with internucleotide linkages, like methylphosphorothioate, phosphodiester, or methylphosphonate can be used. A relatively new technique is conformationally restricted LNA (locked nucleic acid) oligonucleotides and peptide nucleic acids. Bioengineered ribozymes are structurally different, but their specificity also relay on the recognition of the targeted mRNA sequence.

Gene replacement or gene switching techniques inactivate the mutated gene sequence and introduce a corrected one. This can be accomplished by transfecting exogenous gene with normal coding sequence and blocking mutant coding sequence with antisense oligonucleotide. Also a technique with only introducing a corrected normal sequence without disrupting the mutated sequence could be use. This could be used in heterozygous cells i.e. cell carrying one normal allele and one mutated allele resulting in an overexpression of normal alleles. Also homozygous mutant cells could be treated resulting in a dominant positive—effect i.e. the normal allele is expressed in higher degree than the mutant allele.

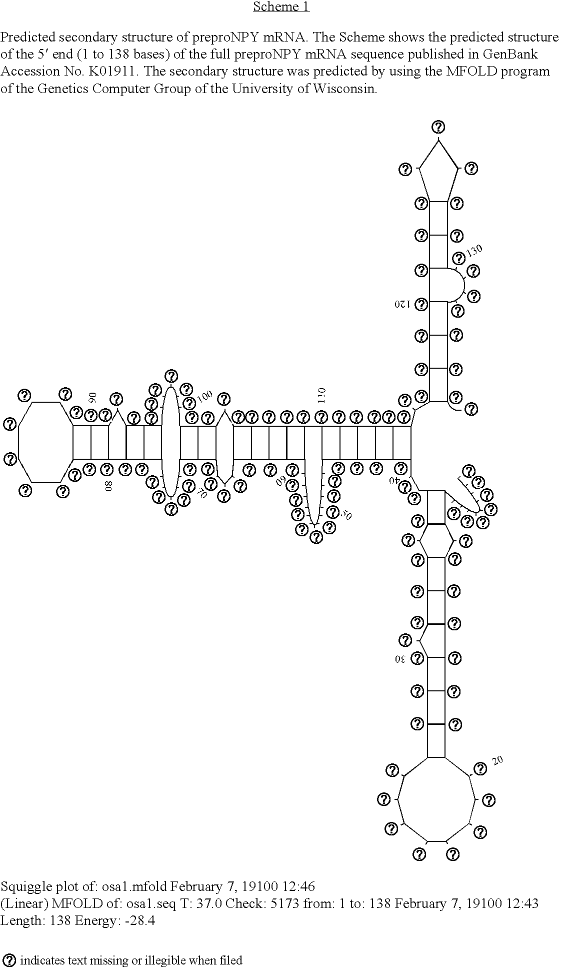

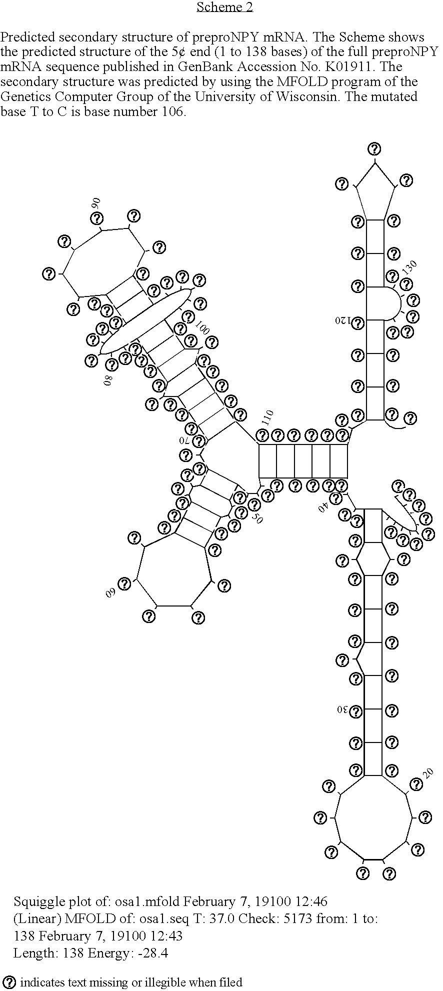

Influence of the mutated NPY sequence on the funtion of NPY gene can be investigated in transgenic animals. A transgenic animal can be generated using targeted homologous recombination methodology. Both normal and mutated sequence of human NPY signal peptide (or any DNA sequence comprising a nucleotide sequence encoding a prepro-neuropeptide Y (preproNPY) or part thereof encoding the amino acid sequence of the mature mouse or human mature NPY peptide, where either i) the leucine amino acid in position 7 of the signal peptide part of said preproNPY has been replaced by proline or ii) the leucine amino acid in position 7 of the signal peptide part of said preproNPY is unchanged) will be introduced into the sequence of NPY gene to replace the endogenous signal peptide sequence. Under these conditions, the endogenous NPY gene functions otherwise normally, but the synthesis of the preproNPY is regulated by either normal or mutated human NPY signal peptide sequence. This transgenic model can be used to investigate in a very specific manner the physiological importance of the mutated NPY gene. It also will provide an ideal preclinical model to investigate and screen new drug molecules, which are designed to modify the influence of the mutated NPY gene.

The invention is described more in detail in the following experiments.

Experimental

Materials and Methods

Study Subjects

The study population consisted of the participants of the Kuopio Ischemic Heart Disease Risk Factor Study (KIHD), a population-based epidemiologic study that was launched in the 1980's to investigate previously unestablished risk factors for myocardial infarction, progression of atherosclerosis, and other major health outcomes in middle-aged men [Salonen, 1988; Lakka et al., 1994]. The study protocol has been approved by the Research Ethics Committee of the University of Kuopio, and all participants gave a written informed consent to participate in KIHD.

The total sample of the KIHD study consists of 2,682 men who were recruited in two cohorts. The present study is based on the second cohort, which is an age-stratified sample of 42-, 48, 54-, and 60 year-old men (N=1,516, participation rate 82.6%) enrolled in the study between 1986 and 1989. A DNA sample was obtained for 1,137 men who were free from coronary heart disease at baseline.

Assessment of Alcohol Consumption

A self-report quantity-frequency questionnaire [Kauhanen et al., 1997a; Kauhanen et al., 1997b] was used to record the level of alcohol use. The average weekly consumption of alcohol in pure ethanol (grams/week) was calculated based on the known alcoholic content of each beverage type and the reported doses and frequencies of drinking sessions. We further calculated the proportion of heavy users consisting of those whose average daily consumption exceeded 3 standard doses (>230 grams of ethanol/week). One dose is a 12 fl ounze bottle of beer, 12 cl of wine, or a 4 cl shot of hard liquor. Serum gamma-glutamyltranspeptidase (GGT) and mean corpuscular volume (MCV) were determined from baseline blood samples as biomarkers of excessive alcohol use. These biochemical measures were checked to see if any of the genotype groups showed biochemical signs of actual alcohol abuse.

Men who told they had not been drinking at all for at least 12 months were determined as abstainers (N=123, a total 12.1%). Since abstainers are a heterogenous group consisting of those who have quit because of health problems, they were excluded from final analyses.

Covariates

A number of sociodemographic, behavioral and medical characteristics were assessed according the KIHD protocol as described earlier [Salonen, 1988; Lakka et al., 1994; Kauhanen et al., 1997a]. Age, place of living (urban/rural), marital status, educational level, current income, history of smoking in cigarette-years, and history of diagnosed chronic diseases and conditions (ischemic heart disease, diabetes, stroke, cancer, liver disease, mental disorder) and history of trauma were recorded by a questionnaire and double-checked in the clinical interview. The data were used to examine the possible effect of confounding in the observed relationship.

Genotype Analysis

PreproNPY genotype was determined by restriction fragment length polymorphism (RFLP) analysis from DNA extracted from the subjects' peripheral blood by an investigator unaware of phenotype. Briefly, the polymorphism appears as a thymidine(1128) to cytosine(1128) substitution generating a Bsi EI restriction site, which was used to genotype the subjects for the Leu7Pro polymorphism, as described previously [Karvonen et al., 1998]. The PCR products were digested by Bsi El [New England Biolabs, Inc. Beverly, Mass., USA] and digestions were analyzed by electrophoresis on 2% agarose gel.

Statistical Analyses

The allelic frequency distribution was tested for Hardy-Weinberg equilibrium by the X²-test. Statistical differences in the mean weekly alcohol consumption between the genotype groups were examined in the analysis of variance. Age and other covariates were adjusted for in analysis of covariance. The proportion of heavy drinkers in the genotype groups was compared using a chi-square test. P-values less than 0.05 obtained from the statistical tests were interpreted as statistically significant. Statistical computations were performed using the SPSS software for IBP RS/6000 [SPSS for Unix, SPSS Inc., Chicago, USA].

Results

The analysis of the Leu(7)-to Pro(7) polymorphism in the signal peptide part of the pre-pro-NPY and complete information on alcohol use was available for 889 alcohol using men. Of these, 790 (88.9%) were genotyped as Leu(7)/Leu(7) homozygous, a total of 95 (10.7%) were Leu(7)/Pro(7) heterozygous, and 4 (0.4%) were Pro(7)/Pro(7) homozygous. The allele frequencies were 94.2% (Leu) and 5.8% (Pro). All men carrying either one or two Pro(7) alleles were pooled for further analyses. The study population was in Hardy-Weinberg equilibrium (chi²=0.585, 1 d.f., p=0.44).

Table I shows sociodemographic and behavioral background characteristics, and the proportion of men with diagnosed diseases in the two NPY genotype groups. There were no differences in the serum level of gamma glutamyl transpeptidase (GGT) or mean corpuscular volume (MCV) between genotypes. The means and standard deviations of GGT were 29.0 U/l (SD 29.4) among Leu(7)/Leu(7) homozygotes and 29.7 U/l (26.0) among those with Pro(7) (p=0.83). For MCV the means and standard deviations were 92.0 fl (SD 4.52) and 92.0 fl (SD 4.0), respectively (p=0.93).

The alcohol consumption in grams of pure ethanol per week is presented in Table II. Both the unadjusted mean consumption and the covariate-adjusted consumption were significantly (33 percent) higher among men who were carriers of Pro(7). The proportion of heavy drinkers (men who reported drinking on average over 230 grams of ethanol/week or over 3 standard doses/day) was also higher among men with a Pro(7) substitution (13.1% vs. 8.2%) (p=0.10).

TABLE I
Means (standard deviations) and proportions of background
variables by the NPY genotype.

| | Leu(7) homozygotes | Pro(7) carriers |
|---|---|---|
| | (N = 790) | (N = 99) |
| Age (years) | 56.1 (SD 6.7) | 56.1 (SD 6.9) |
| Living in rural area | 21.8% | 27.0% |
| Annual income | 24,130 (SD 15,918) | 26,862 (SD 14,771) |
| (US $) | | |
| Educational level | 2.05 (SD 1.75) | 2.13 (SD 1.92) |
| (1 = low, 7 = high) | | |
| Married | 87.1% | 86.9% |
| Cigarette smoking | 141.3 (SD 292.1) | 147.4 (SD 311.7) |
| (pack-years) | | |
| Ischemic heart | 21.1% | 13.1% |
| disease | | |
| Diabetes | 5.6% | 5.1% |
| History of cancer | 2.4% | 5.1% |
| History of stroke | 2.6% | 1.0% |
| Liver disease | 0.4% | 1.0% |
| History of mental | 4.6% | 6.1% |
| disorder | | |
| History of trauma | 10.4% | 10.2% |

TABLE II
Mean weekly alcohol consumption in pure ethanol according to the
NPY genotype.

| | Leu(7) | | |
|---|---|---|---|
| | homozygotes | Pro(7) carriers | |
| | (N = 790) | (N = 99) | P-value |
| Unadjusted mean alcohol | 86.3 (SD 127.6) | 115.0 (SD | 0.030 |
| consumption (g/wk) | | 173.9) | |
| Mean alcohol | 86.4 | 114.7 | 0.035 |
| consumption (g/wk) | | | |
| adjusted for all | | | |
| covariates* | | | |

*Adjusted for age, place of living, education, income, marital status, smoking history in cigarette-years, history of ischemic heart disease, diabetes, cancer, stroke, liver disease, mental disorder and trauma.

Discussion

We observed an increased alcohol consumption in a population sample of middle-aged men who were homozygous or heterozygous for the variant allele in a common polymorphism substituting Leu(7) by Pro(7) in the signal peptide part of neuropeptide Y (NPY). Presence of Pro(7) was associated with approximately one-third (33%) higher average consumption of ethanol as compared to homozygous subjects with the Leu(7)/Leu(7) genotype. The proportion of heavy consumers who report using over 230 grams of ethanol/week was also higher among men with Pro(7) mutation, although this difference did not reach statistical significance due to smaller numbers of subjects.

Our study is the first one to show a relationship between a common NPY polymorphism and alcohol use in humans. The results are in line with the findings from a number of recent animal studies [Ehlers et al., 1998a; Ehlers et al., 1998b; Thiele et al., 1998; Cockerill, 1998; Tecott and Heberlien, 1998] that have shown an inverse relationship between levels of NPY in central nervous system and preference for alcohol. Mice with no neuropeptide Y are especially fond of alcohol and less sensitive to the effects of ethanol as compared to mice that have normal or extra neuropeptide Y levels [Thiele et al., 1998], and alcohol-preferring rats have lower levels of NPY in amygdala, hippocampus, and frontal cortex [Ehlers et al., 1998a].

The allele frequencies in our study were close to those seen earlier in two Finnish populations [Karvonen et al., 1998]. It is highly unlikely that the observed association could be due to a stratification error in sampling, or population admixture, since Finns are known to be genetically a rather homogenous population.

Many sociodemographic factors are known determinants of alcohol use. In our study the social background among men with and without Pro(7) was similar. The two groups were of the same age and had similar educational background. Slightly more men with Pro(7) were living in rural communities, and this group also had a little higher average income. Smoking history was similar in both groups. It was somewhat unexpected to observe a higher prevalence of ischemic heart disease history among the Leu (7) homozygotes, since earlier findings have shown this genotype to associate with lower serum levels of total and LDL choclesterol [Karvonen et al., 1998]. Adjustment for all these variables in the multivariate model did not affect the observed association between the NPY polymorphism and alcohol consumption, indicating that these variables did not confound the findings.

There are several physiologically plausible mechanisms that can explain the effect of NPY on alcohol use. NPY is an inhibitory neuromodulator that acts widely in the brain. The NPY receptors couple to heterotrimeric G proteins that inhibit production of cyclicAMP [Thiele et al., 1998; Lamme, 1995], so it is possible that NPY inhibits cAMP production in response to alcohol, thus limiting alcohol intake. Central administration of NPY reduces anxiety, and NPY-deficient mice score high on measures of anxiety [Heilig et al., 1992; Palmiter et al., 1998]. The development of alcohol preference may in part depend on the relative lack of tension-reducing NPY.

Chronic exposure to ethanol in rats affects NPY levels in hypothalamus in a fashion similar to food restriction [Ehlers et al., 1998a]. NPY has an important role in the hypothalamic regulation of energy balance by potently stimulating short-term food intake [Clark et al., 1985; Levine and Morley, 1985; Stanley and Leibowitz, 1985]. Centrally administered NPY also increases the expression of lipoprotein lipase mRNA and enhances the enzyme activity in white fat favoring lipid storage [Billington et al., 1991; Billington et al., 1994]. Thus, NPY might unspecifically affect the consummatory behaviors regarding both food intake and alcohol drinking. However, there is a lack of NPY transgene expression in the arcuate nucleus of the hypothalamus, a region thought to regulate food intake [Thiele et al., 1998; Palmiter et al., 1998]. This indicates that the effects of NPY on alcohol use are probably not mediated through similar mechanisms as with food and calorie intake.

To our knowledge, there is only one earlier human study examining the possible relationship between neuropeptide Y and addictions. Roy and coworkers [1990] did not observe significant differences of cerebrospinal fluid (CFS) levels of NPY between male alcoholics and normal controls. Alcoholics, however, do not represent the population at large. It is also unclear, whether the CFS levels of NPY reflect the activity of this peptide in the physiologically important locations of the central nervous system.

Plasma NPY is derived from sympathetic nerve terminals and thus levels of NPY in plasma reflect the level of sympathetic activity [Lundberg et al., 1990]. Significant positive correlations have been observed between levels of NPY and corticotropin-releasing hormone, somatostatin, and growth hormone in cerebrospinal fluid [Roy et al., 1990]. Based on these studies and on our findings, further research on the possible sympathetic nervous system mechanisms in drinking behavior is warranted.

It will be appreciated that the methods of the present invention can be incorporated in the form of a variety of embodiments, only a few of which are disclosed herein. It will be apparent for the specialist in the field that other embodiments exist and do not depart from the spirit of the invention. Thus, the described embodiments are illustrative and should not be construed as restrictive.

### REFERENCES

Ault D T, Radeff J M, Werling L L. 1998. Modulation of (3H)dopamine release from rat nucleus accumbens by neuropeptide Y may involve a sigma 1-like receptor. J Pharmacol Exp Ther 284:553-560.

Billington C J, Briggs J E, Grace M, Levine A S. 1991. Effects of intracerebroventricular injection of neuropeptide Y on energy metabolism. Am J Physiol 260: R321-R327.

Billington C J, Briggs J E, Harker S, Grace M, Levine A S. 1994. Neuropeptide Y in hypothalamic paraventricular nucleus: a center coordinating energy metabolism. Am J Physiol 266: R1765-R1770.

Clark J T, Kalra P S, Kalra S P. 1985. Neuropeptide Y stimulates feeding but inhibits sexual behavior in rats. Endocrinology 117:2435-2442.

Cockerill M. 1998. Low levels of brain chemicals drives mice to drink. Brit Med J 317:1544.

Ehlers C L, Li T K, Lumeng L, Hwang B H, Somes C, Jimenez P, Mathe A A. Neuropeptide Y levels in ethanol-naive alcohol-preferring and nonpreferring rats and in Wistar rats after ethanol exposure. 1998a. Alcohol Clin Exp Res 8:1778-1782.

Ehlers C L, Somes C, Cloutier D. 1998b. Are some of the effects of ethanol mediated through NPY? Psychopharmacology 139:136-144.

Gray T S, Morley J E. 1986. Neuropeptide Y: anatomical distribution and possible function in mammalian nervous system. Nature 38:389-401.

Heilig M, McLeod S, Koob G K, Britton K T. 1992. Anxiolytic-like effect of neuropeptide Y (NPY), but not other peptides in an operant conflict test. Regul Pept 41:61-69.

Jewett D C, Cleary J, Levine A S, Schaal D W, Thompson T. 1992. Effects of neuropeptide Y on food-reinforced behavior in satiated rats. Pharmacol Biochem Behav 42:207-212.

Karvonen M K, Pesonen U, Koulu M, Niskanen L, Laakso M, Rissanen A, Dekker J M, Hart L M, Valve R, Uusitupa M I J. 1998. Association of a leucine(7)-to-proline(7) polymorphism in the signal peptide of neuropeptide Y with high serum cholesterold and LDL cholesterol levels. Nature Med 4:1434-1437.

Kauhanen J, Kaplan G A, Goldberg D E, Salonen J T. 1997a. Beer binging and mortality: results from the Kuopio ischaemic heart disease risk factor study, a prospective population based study. Brit Med J 315:846-851.

Kauhanen J, Kaplan G A, Goldberg D, Cohen R D, Lakka T A, Salonen J T. 1997b. Frequent hangovers and carciovascular mortality in middle-aged men. Epidemiology 8:310-314.

Lakka T A, Venäläinen J M, Rauramaa R, Salonen R, Tuomilehto J, Salonen J T. 1994. Relation of leisure-time physical activity and cardiorespiratory fitness to the risk of acute myocardial infarction in men. N Engl J Med 330:549-1554.

Lamme V A F. 1995. The neurophysiology of figure-ground segregation in primary visual cortex. J Neurosci 15:1605-1615.

Levine A S, Morley J E. 1985. Neuropeptide Y: a potent inducer of consummatory behavior in rats. Peptides 5:1025-1029.

Lundberg J M, Terenius L, Hokfelt T, Martling C R, Tatemoto K, Mutt V, Polak J, Bloom S, Goldstein M. 1982. Neuropeptide Y (NPY)-like immunoreactivity in peripheral noradrenergic neurons and effects of NPY on sympathetic function. Acta Physiol. Scand 116:477-480.

Lundberg J M, Franco-Cereceda A, Hemsen A, Lacroix J S, Pernow J. 1990. Pharmacology of noradrenaline and neuropeptide tyrosine (NPY)-mediated sympathetic cotransmission. Fundam Clin Pharmacol 4:373-391.

Palmiter R D, Erickson J C, Hollopeter G, Baraban S C, Schwartz M W. 1998. Life without neuropeptide Y. Recent Prog Horm Res 53:163-199.

Roy A, Berrettini W, DeJong J, Adinoff B, Ravitz B, Linnoila M. 1990. CSF neuropeptide Y in alcoholics and normal controls. Psychiatry Res 33: 215-219.

Salonen J T. 1988. Is there a continuing need for longitudinal epidemiologic research? The Kuopio ischemic heart disease risk factor study. Ann Clin Res 20:46-50.

Stanley B G, Leibowitz S F 1985. Neuropeptide Y injected in the paraventricular hypothalamus: a powerful stimulant of feeding behavior. Proc Natl Acad Sci USA 82: 3940-3943.

Stewart R B, Gatto G J, Lumeng L, Li T-K, Murphy J M. 1993. Comparison of alcohol-preferring (P) and nonpreferring (NP) rats on tests of anxiety and for the anxiolytic effects of alcohol. Alcohol 10:1-10.

Tecott L H, Heberlein U. 1998. Y do we drink? Cell 95:733-735.

Thiele T E, Marsh D J, Ste Marie L, Bernstein I L, Palmiter R D. 1998. Ethanol consumption and resistance are inversely related to neuropeptide Y levels. Nature 396:366-369.

1. A method for diagnosing a person's susceptibility for having a risk for the development of alcoholism, said method comprising determining whether said subject has a polymorphism in the signal peptide part of the human preproNPY, said polymorphism comprising the subsitution of the position 7 leucine for proline in the signal peptide part of said preproNPY, said polymorphism being indicative of a risk for the development of alcoholism.

2. The method according to claim 1 wherein said polymorphism in the signal peptide part of the human preproNPY at said subject is determined by subjecting a position 7 allele specific oligonucleotide probe to a sample from said subject, said sample comprising a target polynucleotide of said preproNPY.

3. The method according to claim 1 wherein said polymorphism in the signal peptide part of the human preproNPY at said subject is determined by immunoassay where a sample from said subject is contacted with an antibody capable of binding the signal peptide or said NPY peptide associated with any other cleavage product of preproNPY.

4. A method for treating a person, diagnosed for having a risk for the development of alcoholism according to claim 1, 2 or 3, for the prevention of developing alcoholismor for alleviating or curing alcoholism, comprising administering to said person an effective amount of an agent counteracting the influence of the mutated NPY gene.

5. The method according to claim 4 wherein said agent is a pharmaceutical aimed to modulate synthesis, secretion or metabolism of the endogenous NPY, or to interact in a specific manner at NPY target sites by modulating effects of NPY with specific NPY receptor proteins.

6. The method according to claim 4 wherein said agent is a pharmaceutical aimed to modulate gene expression of normal or mutated NPY gene.

7. A method for treating a person, diagnosed for having a risk for the development of alcoholism according to claim 1, 2 or 3, for the prevention of developing alcoholism or for alleviating or curing alcoholism, comprising subjecting the person to specific gene therapy aimed to repair the mutated NPY sequence.

8. A method to investigate or screen pharmaceuticals or genetic aims useful in the prevention or treatment of alcoholism, by using an animal model including a transgenic animal which carries a human DNA sequence comprising a nucleotide sequence encoding a prepro-neuropeptide Y (preproNPY) or part thereof encoding mature human NPY peptide, where the leucine amino acid in position 7 of the signal peptide part of said preproNPY i) is unchanged or ii) has been replaced by proline.

9. A method to investigate or screen pharmaceuticals or genetic aims useful in the prevention or treatment of alcoholism, by using an animal model including a transgenic animal, which carries a DNA sequence comprising a nucleotide sequence encoding otherwise normal mouse NPY sequence or part thereof encoding mature mouse NPY peptide, but in which the nucleotide sequence encoding the mouse signal peptide is replaced by human signal peptide sequence encoding either normal or mutated human signal peptide.
